(19) United States
(12) Patent Application Publication
(10) Pub. No.: US 2023/0393126 A1
(43) Pub. Date: Dec. 7, 2023

(54) METHOD FOR EVALUATING THE MECHANOBIOLOGICAL PROPERTIES OF A CELL ELEMENT

(71) Applicants: CENTRE NATIONAL DE LA RECHERCHE SCIENTIFIQUE, Paris (FR); UNIVERSITÉ PARIS CITÉ, PARIS (FR)

(72) Inventors: NICOLAS MINC, PARIS (FR); JEREMY SALLE, BOULOGNE BILLANCOURT (FR); JING XIE, PARIS (FR); JAVAD NAJAFI, LE KREMLIN-BICÊTRE (FR)

(21) Appl. No.: 18/032,817

(22) PCT Filed: Oct. 20, 2021
(86) PCT No.: PCT/EP2021/079072

§ 371 Date: Apr. 20, 2023

(30) Foreign Application Priority Data
Oct. 20, 2020 (FR) 2010766

(51) Int. Cl. G01N 33/543

(52) U.S. Cl. CPC G01N 33/54326

(57) ABSTRACT

The present invention concerns a method for characterizing the mechanobiological properties of a cell element or compartment, said method relying on the use of a magnetic tip and superparamagnetic beads.

### TECHNICAL FIELD

The present invention relates to the field of biology, in particular mechanobiology, and more particularly concerns a method for characterizing the mechanobiological properties of a cell or of one of its elements or compartments.

### TECHNOLOGICAL BACKGROUND

Mechanobiology is an emerging field of research aimed at understanding how the mechanics of cellular organelles, cells, tissues, and/or their environment influence the physiology of organisms. One of the foundations of this field of research and its biomedical applications is to be able to accurately measure and precisely apply mechanical forces to the surface of organelles and cells in complex tissue environments.

Mechanical forces within and around cells can influence key processes such as differentiation, division or cell migration (Engler et al., 2006; Ladoux and Mège, 2017). Importantly, many diseases such as cancer, cardiovascular disease, genetic diseases and developmental malformations are typically associated with drastic changes in the mechanical properties of cells and tissues (Carey et al., 2012; Makale, 2007; Murthy et al., 2017). Within cells, the nucleus also has a surface shape and stiffness, essential for the integrity of the nuclear envelope and the preservation of genetic information. Thus, the shape, size and mechanics of the nucleus are very often used as markers of cancer progression (Jevtic and Levy, 2014).

These recent discoveries have led to the development of a new multidisciplinary discipline, mechanobiology, at the interface between engineering, biology and medicine, which consists of studying the regulation and role of nuclear, cellular and tissue forces to characterize the behaviour of living systems and their deregulation in different diseases (Lim et al., 2010). Mechanobiology involves the application and measurement of forces on living cells and/or tissues (Roca-Cusachs et al., 2017). Currently, two commercial methods are available: (i) optical tweezers and (ii) atomic force microscopy.

The optical tweezer method is a method proposed for applying forces or probing the mechanical properties of the surface or interior of cells. This method has a range of forces typically comprised between 0.01 pN and 10 pN, and applies to molecular issues (single biological filaments or motors). In return, it is unsuitable for volume measurements, since the force becomes too low as soon as one moves away from the focal plane of the laser and its range of forces does not correspond to the typical cellular and tissue forces (10-1000 pN). This method also requires the use of expensive infra-red lasers whose alignment must be systematically calibrated, often involving long and user-dependent protocols.

The atomic force microscopy (AFM) method consists of indenting the surface of cells or tissues to measure their stiffness, for example, by means of a tip coupled to a calibrated deflector. It cannot be applied to measurements within the volume of cells or tissues and creates substantial damage to cells, especially in the ranges of forces useful for studying the mechanobiology of a cell. It is therefore limited in its compatibility with living samples. It is also associated with a complex assembly, requiring precise calibrations that are not always agreed upon in the community (different models of interpretation of distance-force curves).

Thus, the methods currently available are largely limited to the application of forces on thin samples, and the range of forces usable in these techniques remains limited to a few piconewtons (pN) at most on living samples. They are also expensive and difficult to implement on a routine basis, and poorly compatible with dynamic imaging.

Thus, there is a need to develop new strategies for studying and characterizing cellular mechanobiological properties and, in particular, to provide a method making it possible to have precise information on cellular and tissue biomechanics that can be used in a wide range of applications.

### SUMMARY OF THE INVENTION

The invention aims to overcome the lack of robust, versatile and precise methods of application and force measurements in biological samples. The inventors propose here a method based on the use of magnetic tips adapted to tissue, cellular and nuclear surface mechanics, allowing forces to be applied in ranges from pN to nN, in deep tissues or in developing multicellular non-human embryos. This method relies on magnetized micro-tips that apply magnetic forces to functionalized paramagnetic microparticles to bind specifically to cell elements and/or compartments. This approach especially allows localized applications of forces: (i) within cells, for applications, for example, for the study of the mechanics of the nucleus (ii) on the surface of a single cell or within large multicellular tissues or embryos (iii) with a range of forces exceeding by 2-3 orders of magnitude the range of common commercial methods such as optical tweezers and atomic force microscopy and/or (iv) with optimal compatibility with living cells and tissues. Here, the inventors demonstrate the versatility, robustness and applicability of the method according to the invention in invertebrate and vertebrate cells and tissues, in the context of a deformation of the nucleus within a living cell and application of surface force on single cells as well as within the lumen of large multicellular assemblies.

The method has been validated in several cell types and developing multicellular embryos. These include embryos of echinoderms (sea urchins), insects (fruit flies), vertebrates (zebrafish) and mammalian cells in culture.

The two main competing techniques for probing the mechanics of cells and tissues are optical tweezers, and atomic force microscopy. With regard to the invention, these methods are: (i) limited to surface measurements of thin samples (adherent or monolayer cells), (ii) limited to a low range of forces that are poorly compatible with the forces required to study most cells and tissues, (iii) invasive, and therefore poorly compatible with the physiology and imaging of living cells over long periods, (iv) very difficult to implement and (v) much more expensive.

The invention differs from other methods of measuring and applying forces by its versatility in terms of applications to study mechanobiological properties concerning cellular organelles, properties of the cellular cytoplasm and surface forces within single cells or cells inserted into complex and deep tissue. In addition, this approach is fully compatible with living samples and with the development and proliferation of living cells and tissues and allows for a much wider range of force than optical tweezer methods.

Beyond information on the physiology of many tissues and embryos, the versatility and simplicity of implementation of the method promises an application in industrial biomedical research contexts. For example, the invention can serve as a standard for rapid and accurate classification of the mechanical properties of tissues in the context of disease diagnostics, for establishing rapid tests to screen the mechanobiological responses of healthy and diseased organs and tissues, to establish mechanical signatures useful in the diagnosis of unhealthy tissues in the field of health, or to develop chemical screens specifically related to tissue mechanobiology, in particular to evaluate the effect of drugs or small molecules on mechanobiological responses (stiffness of a healthy or tumour tissue or cell, structural properties of the nucleus, etc.).

The invention relates to an in vitro or ex vivo method for characterizing at least one mechanobiological property of at least one element, at least one cell compartment, at least one cell or at least one group of cells in a biological sample, said method comprising:
a) introducing superparamagnetic beads into the biological sample, said beads comprising a coating capable of targeting the superparamagnetic beads to a cell element or compartment of interest or of penetrating a cell or group of cells,
b) applying a magnetic field to the superparamagnetic beads in order to apply a force to the biological sample,
c) measuring at least one mechanobiological property of the cell element or compartment in the biological sample,
   said biological sample being a cell or a multicellular biological sample and
   the magnetic field being applied by a magnetic tip, said magnetic tip comprising at least one millimetre or submillimetre sized magnet extended by a metal tip.

According to one aspect, the coating is a hydrophobic coating. In particular, superparamagnetic beads comprising a hydrophobic coating are mixed with a biocompatible oil capable of causing the superparamagnetic beads to penetrate into a cell or a group of cells, before being introduced into the biological sample.

According to one aspect, the coating is a biological coating capable of specifically targeting the superparamagnetic beads to a cell element or compartment of interest, a cell or a group of cells. In particular, the superparamagnetic beads comprising a biological coating are mixed with a solution, preferably a buffer solution, before being introduced into the biological sample.

Preferably, said biological sample is a multicellular biological sample chosen from: a tissue, an organ or an embryo, said biological sample not being of human embryonic origin. In particular, the cell element or compartment is chosen from: the cell membrane, the nuclear envelope, the membrane of an organelle, such as mitochondria, endoplasmic reticulum or Golgi apparatus, lysosomes, peroxisomes and their contents, such as cytoplasm, microtubules and/or centrioles, centrosomes or mitotic spindle, preferably the cell membrane or nuclear envelope. In particular, the cell element or compartment is the internal or external cell membrane of at least one cell of the biological sample.

According to one aspect, the force applied to the biological sample is between 0.001 pN and 5000 nN, between 0.001 pN and 1000 nN, between 0.001 pN and 20 nN, between 0.001 nN and 5000 nN, between 0.01 nN and 1000 nN, between 0.001 pN and 20 nN, or between 10 nN and 1000 nN, preferably between 0.001 pN and 100 nN, preferentially between 0.5 pN and 1.5 nN.

Preferably, the distance between the magnetic tip and the biological sample is comprised between 10 micrometres and 1 millimetre.

The metal tip is, in particular, machined with a radius of curvature comprised between 5 and 100 micrometres, preferably between 5 and 50 micrometres. The metal tip preferably has a length comprised between 1 and 30 millimetres, for example between 10 and 20 millimetres.

According to one aspect, the method further comprises (i) a preliminary step in which the biological coating of the beads is selected according to the cell element or compartment of interest, (ii) a preliminary step of preparing the tip and/or superparamagnetic beads and/or (iii) a step of calibrating the tip and beads.

According to another aspect, the method further comprises (i) a preliminary step of preparing the tip and/or superparamagnetic beads, (ii) a preliminary step in which superparamagnetic beads comprising the hydrophobic coating are mixed with a biocompatible oil, and/or (iii) a step of calibrating the tip and the beads.

Preferably, the mechanobiological properties are chosen from: rheology, kinematics, mechanical strength, elasticity, stiffness, plasticity and viscoelastic properties, and any combination thereof.

According to one aspect, the coating of the superparamagnetic beads is a hydrophobic coating chosen from butyl, methyl, ethyl, octyl, propyl or octadecyl, preferably octadecyl. Preferably, the biocompatible oil is chosen from a soya oil, a linseed oil, an oil of the (C15-C40) alkane type, and a fluorocarbon oil.

According to another aspect, the biological coating of the superparamagnetic beads is a protein coating, comprising at least one bait protein capable of recognizing a prey protein on the cell element or compartment of interest. Preferably, the biological coating of the superparamagnetic beads is an antibody or a lectin. Preferentially, the biological coating of the superparamagnetic beads is an antibody chosen from an anti-sodium potassium ATPase, anti-PMCA1, anti-Pan-cadherin, anti-E-cadherin, anti-N-cadherin, anti-ADAM22, anti-LRP4, anti-Fas (APO-1, CD95), anti-CD27, anti-EGFR, anti-NPC and anti-NUP98 antibody. The solution for suspending these beads is preferably a buffer solution, preferably a phosphate buffer.

Preferably, the superparamagnetic beads have a size comprised between 100 nm and 100 μm, preferably between 100 nm and 50 μm, preferentially between 100 nm and 1 μm.

According to one aspect, the superparamagnetic beads comprise a biological coating and in step a) of the method according to the invention, the superparamagnetic beads are injected into a cell compartment of the biological sample.

In particular, the superparamagnetic beads are introduced by microinjection, biolistics or bombardment, preferably by microinjection, preferentially by pneumatic or hydraulic microinjection.

The biological sample may be obtained beforehand by sampling. The biological sample may be living and/or unfixed.

The methods of the invention make it possible to (i) characterize the impact of a drug or medicament on a biological sample, and/or (ii) identify tumour cells in a biological sample.

In particular, the methods according to the invention make it possible to (i) characterize the impact of a drug or a medicament on a cell, a tissue, an organ or an embryo or one of its elements or compartments, and/or (ii) identify and characterize damaged cells or tissues and/or (iii) identify and characterize tumour cells in a sample, said tumour cells exhibiting mechanobiological characteristics different from normal cells, and/or (iv) study the mechanical interaction of cells with each other or with the surrounding matrix and/or (v) study the mechanotransduction of at least one cell.

The invention also relates to a kit comprising:
a) a magnetic tip comprising a magnetic core comprising a superposition of millimetre or submillimetre sized magnets extended by a steel tip,
b) superparamagnetic beads comprising a coating capable of targeting the superparamagnetic beads to a cell element or compartment of interest or penetrating a cell or group of cells,
c) optionally, a solution allowing to suspend and inject the superparamagnetic beads into a cell or cell tissue and
d) optionally, at least one solution for calibrating the superparamagnetic beads and the magnetic tip.

Which can be used, in particular, to characterize at least one mechanobiological property of at least one element, at least one cell compartment, at least one cell or at least one group of cells in a biological sample,

In this kit, the coating can be a hydrophobic coating, preferably an octadecyl, and the solution is a biocompatible oil capable of causing the superparamagnetic beads to penetrate into a cell or a group of cells. Alternatively or additionally, the kit may comprise superparamagnetic beads comprising a biological coating capable of specifically targeting the superparamagnetic beads to a cell element or compartment of interest, the coating of the superparamagnetic beads preferably being a protein coating, preferably an antibody or a lectin and the solution being a buffer solution, preferably a phosphate buffer.

In the kit according to the invention, the end of the steel tip is machined with a radius of curvature comprised between 5 and 100 micrometres. The radius of curvature of the steel tip is preferably configured to apply a uniaxial magnetic gradient to the biological sample. In particular, the magnetic core and the end of the steel tip are configured to allow a force to be applied to the biological sample comprised between 10 nN and 1000 nN, for a distance between the magnetic tip and the biological sample comprised between 10 micrometres and 1 millimetre. Preferably, the steel tip has a length comprised between 1 and 30 mm.

According to one aspect, the magnet of the magnetic core is made of neodymium, ticonal, alnico, neodymium iron boron (NdFeB) or samarium cobalt (SmCo), preferably neodymium.

According to one aspect, the steel tip is covered with a layer of gold.

The kit according to the invention may also comprise a microinjector.

The invention also concerns the use of the kit according to the invention, for (i) characterizing the impact of a drug or a medicament on a biological sample, in particular a cell, a tissue, an organ or a non-human embryo or one of its elements or compartments, and/or (ii) identifying and characterizing damaged cells or tissues and/or (iii) identifying and characterizing tumour cells in a sample, said tumour cells having, in particular, mechanobiological characteristics different from normal cells, and/or (iv) studying the mechanical interaction of cells with each other or with the surrounding matrix and/or (v) studying the mechanotransduction of at least one cell.

In particular, the use of the kit makes it possible to (i)—determine the mechanical properties of the surface of the nucleus, within a living cell, such as the elasticity of the nuclear envelope; (ii) determine the stiffness of the cell surface, such as the viscoelastic properties of the cortex; (iii) apply forces within tissues to depths of around 100 micrometres, and measure, for example, mechanical constants such as mechanical constants of compressive stiffness and surface elasticity; and/or (iv) perform measurements in a volume of deep samples such as developing embryos, tissue explants and organoids.

The invention also relates to an in vitro or ex vivo method for characterizing at least one mechanobiological property of at least one element, at least one cell compartment, at least one cell or at least one group of cells in a biological sample, said method comprising:
a) introducing superparamagnetic beads into the biological sample, said beads comprising a coating capable of specifically targeting the superparamagnetic beads to a cell element or compartment of interest,
b) applying a magnetic field to the superparamagnetic beads in order to apply a force to the biological sample,
c) measuring at least one mechanobiological property of the cell element or compartment in the biological sample,
characterized in that
said biological sample is a cell, a multicellular tissue or embryo, or an organ, and said biological sample is not a biological sample of human embryonic origin
   the magnetic field is applied by a magnetic tip, said magnetic tip comprising at least one millimetre or submillimetre sized magnet extended by a metal tip.

The invention also relates to a method, preferably in vitro or ex vivo, for characterizing at least one mechanobiological property of a cell element or compartment in a biological sample, said method comprising:
a) introducing superparamagnetic beads into the biological sample, said beads comprising a biological coating capable of specifically targeting the superparamagnetic beads to a cell element or compartment of interest,
b) applying a magnetic field to the superparamagnetic beads in order to apply a force to the biological sample,
c) measuring at least one mechanobiological property of the cell element or compartment in the biological sample.

### DESCRIPTION OF THE DRAWINGS

FIG. 1 shows the magnetic tip principle for applying force to the surface of the nucleus, single cells and multicellular tissues. A. Diagram showing the magnetic tip device: A permanent magnet extended by a metal tip is mounted on a 3D micromanipulator. The tip is brought close to a living sample placed in a culture medium and observed under a microscope. B. Schematic example of the application of force to a cell culture containing magnetic particles specifically targeted to the plasma membrane or nuclear envelope. C. Schematic example of the application of forces to a tissue containing magnetic particles associated with the plasma membrane or trapped in the lumen of the epithelium.

FIG. 2 shows the calibration of the applied magnetic forces.

FIG. 2A The magnetic beads placed in a viscous medium move towards the tip and their instantaneous speed (represented with a grey gradient) makes it possible to calibrate the force as a function of the distance from the magnetic tip.

FIG. 2B Magnetic force created by the tip on magnetic particles (1 m diameter) in media of different viscosities (viscosity indicated in the legend). Viscosity modulation allows for more accurate force determination over a wider range of distances.

FIG. 3 shows the application of forces within cells and embryos and effect(s) on cell division and development.

FIG. 3A Displacement of magnetic particles within a living sea urchin cell.

FIG. 3B Displacement of magnetic beads within a fruit fly embryo.

FIG. 3C Successive divisions of sea urchin cells injected with fluorescent beads.

FIG. 3D Application of forces to the single epithelial cell surface within fruit fly embryonic tissue injected with magnetic beads.

FIG. 4 shows the measurement of the stiffness of the nucleus within an intact cell. Magnetic beads covered with antibodies directed against nuclear surface proteins (anti-NPC), injected into a living sea urchin cell, bind specifically to the nuclear surface and allow the nucleus to be pulled and deformed. The deformation and the value of the applied force make it possible to calculate the stiffness of the nuclear surface.

FIG. 5 shows the measurement of the stiffness of the surface of a single cell. Magnetic beads were injected into a sea urchin embryo at stage 1 of development. The presence of the magnet causes the aggregation of the magnetic beads and the deformation of the cell membrane. The deformation and the value of the force applied (here ≈660 pN) make it possible to calculate the stiffness of the cellular surface.

FIG. 6 shows the measurement of the mechanical properties of a tissue in a 3D multicellular system. A large magnetic bead injected into the heart endocardium of a living zebrafish allows calibrated forces to be applied to the surface tissues and the compression and deformation stiffness of the tissues to be calculated.

FIGS. 7A and B show examples of magnetic tips according to the invention.

FIG. 8 illustrates a method for displacing large drops of oil into the cytoplasm of a cell to probe the mechanobiological properties of the cytoplasm. A. Diagram where hydrophobic beads are placed in a drop of oil injected into the cytoplasm. B. Calibration of the force applied to the oil drop according to the size of the aggregate. C. Example of an experiment where the drop of oil is displaced by the application of a force (red zone) and then returns to its original position when the force is released, due to the visco-elastic properties of the cytoplasm of a sea urchin zygote cell.

### DETAILED DESCRIPTION OF THE INVENTION

The method according to the invention is based on the use of superparamagnetic beads and magnetic tips to evaluate the mechanobiological characteristics of at least one cell, element, or compartment thereof. In particular, the method according to the invention makes it possible to evaluate at least one mechanobiological property of at least one element, at least one cell compartment, at least one cell or at least one group of cells in a biological sample. These various elements are more particularly described below.

### Superparamagnetic Beads

The method according to the invention is compatible with all kinds of superparamagnetic particles or beads. There are a large number of them commercially available, with a range of surface chemistry that can be varied. The inventors tested and validated the method according to the invention with a dozen beads of different brands, without any sign of defect or limit of the approach.

As used here, the term “paramagnetism” refers to the behaviour of a material that does not have spontaneous magnetization but that, under the effect of an external magnetic field, acquires magnetization oriented in the same direction as the applied magnetic field, for example a field as applied by a magnetic tip. A paramagnetic material has a positive magnetic susceptibility. The magnetization of these materials can also reverse spontaneously under the influence of temperature, especially at the Curie temperature of the material. An exchange interaction between magnetic moments exists which, below a certain temperature, leads to a magnetic order (ferromagnetic, ferrimagnetic or antiferromagnetic). Above this temperature, the moments fluctuate and these materials are characterized by a positive scalar magnetic susceptibility of around 10⁻⁵ to 10⁻³. The term ‘supraparamagnetism’ or ‘superparamagnetism’ is understood to mean the behavior of paramagnetic materials of micrometric or nanometric dimensions.

According to a particular embodiment, the superparamagnetic beads consist of cores of iron oxides such as magnetite, of micrometric or nanometric size, and generally coated in a polymer envelope. The magnetic moment of the beads depends on the number of cores or magnetic domains. Beads formed by a large number of domains have a strong magnetic moment and are therefore easily attracted by a permanent magnet. As they are superparamagnetic, the beads quickly acquire magnetization under the effect of a magnetic field. This magnetization disappears immediately when the field is removed.

Such beads are sold in particular by Invitrogen (Dynabeads™ MyOne™ Streptavidin C1), Chemicell (nano-screenMAG-Streptavidin), Promega (High Capacity Magne® Streptavidin Beads), Nvigen (MyQuVigen™—Streptavidin) and Solulink (NanoLink™ Streptavidin Magnetic Beads).

According to one embodiment, the superparamagnetic core of the beads is included in a layer of polymer (e.g., polysaccharides) functionalized with a binding domain (e.g., streptavidin). This binding domain is used for the attachment of the coating which will ensure that the beads are targeted to a particular cell element or compartment.

‘Targeting’ is understood here as a mechanism allowing the transport of superparamagnetic beads to their intra- or extracellular destination of interest. For example, when the beads have a coating consisting of anti-cadherin antibodies, a plasma membrane protein, the beads are targeted (i.e., transported/localized and then fixed) to the plasma membrane after their injection.

The term ‘coating’ is understood here to mean a layer deposited on the surface of a material, here a superparamagnetic bead, in order to impart particular properties to it. In particular, the coating present on the surface of the paramagnetic beads allows the beads to be specifically targeted to a cell compartment or element and possibly attached to it. The coating may, in particular, be a biological coating, such as a protein, peptide or lipid coating or a hydrophobic coating.

According to one aspect, the coating is a hydrophobic coating, in particular chosen from long carbon chains such as butyl, methyl, ethyl, octyl, propyl, or octadecyl.

Preferably, the hydrophobic coating is composed of octadecyl-type carbon chains. Preferably, when the superparamagnetic beads comprise a hydrophobic coating, the beads are mixed with an oil to be injected into the cytoplasm of the cells of the biological sample. The oils which can be used may be organic or mineral and are preferably biocompatible. For example, the oils may be chosen from a soya bean oil, a linseed oil, an oil of the (C15-C40) alkane type, or a fluorocarbon oil.

According to another aspect, the beads comprise a biological coating and are shaped to be attached to the inner face of the biological sample, to the nuclear envelope of a cell of the biological sample, to the mitotic spindle and/or to the centrosome of the biological sample. Preferably, the beads are prepared so as to allow their attachment to the centrosome of the biological sample.

Preferably, the cell compartment or element is chosen from: the cell membrane, the nuclear membrane, the membrane of an organelle, such as the mitochondria, the endoplasmic reticulum or the Golgi apparatus, the lysosomes, the peroxisomes and their contents, such as the cytoplasm, the microtubules and/or the centrioles.

Preferably, the coating present on the surface of the beads is a biological coating which allows them to be targeted and attached to the cell membrane or to the nuclear membrane, or to other cell organelles.

Preferably, the beads are attached to a cell compartment or element by non-covalent bonding, especially a protein-protein type bond, in particular of the bait protein-prey protein type, or of the antibody-antigen type.

The term ‘prey protein’ here is understood to mean a cellular protein of interest, which a person skilled in the art would like to target in order to evaluate the mechanobiological characteristics of a cell, a compartment or an element thereof.

The term ‘bait protein’ or ‘hook protein’ is understood here to mean a protein capable of recognizing, interacting and binding with the prey protein, in particular having a specific affinity for a cell compartment/structure.

The person skilled in the art knows how to identify bait protein-prey protein pairs of interest. For example, to specifically target the superparamagnetic beads to the nuclear membrane, a prey protein may be a protein constituting the nuclear pores and the bait protein an antibody specifically recognizing this protein of the nuclear pores.

According to one embodiment, the coating of the superparamagnetic beads is a protein coating, preferably an antibody, peptide or lectin.

Magnetic beads functionalized with proteins such as antibodies are commercially available. Alternatively, functionalization methods and kits are available to coat the beads with a compound of interest. These methods and kits are well known to the skilled person. For example, the reaction between the sulfonic ester and carboxylic acid groups makes it possible to create covalent bonds between the beads and the compound of interest, while the beads-streptavidin are functionalized via the biotin-streptavidin interaction by antibodies or other proteins previously covalently coupled to biotin.

For antibodies that are not available in biotinylated version, their prior biotinylation is possible, especially via the use of a biotinylation method and kit, for example the EZ-link NHS-biotin kit marketed by Sigma.

According to one embodiment, the coating of the super-paramagnetic beads is an antibody, preferably chosen from an anti-sodium potassium ATPase, anti-PMCA1, anti-Pan-cadherin, anti-E-cadherin, anti-N-cadherin, anti-ADAM22, anti-LRP4, anti-Fas (APO-1, CD95), anti-CD27, anti-EGFR, anti-NPC and anti-NUP98 antibody.

Preferably, the ‘hook’ proteins used to specifically target the particles to the plasma membrane or nuclear envelope are chosen from the following table:

TABLE 1

| Hook proteins | Location | Reference |
|---|---|---|
| Anti-sodium-potassium | plasma membrane | Abcam (ab76020) |
| ATPase | | |
| antibodies | | |
| Anti-PMCA1 antibody | plasma membrane | Abcam(ab3528) |
| Anti-Pan-cadherin antibody | plasma membrane | Abcam (ab6529) |
| Anti-E-cadherin antibody | plasma membrane | Abcam (ab1416) |
| Anti-CD98 antibody | plasma membrane | Abcam (ab23495) |
| Anti-Ezrin antibody | plasma membrane | Abcam (ab40839) |
| Anti-Zonula occludens protein | plasma membrane | Abcam (ab181991) |
| 3/zo3 antibody | | |
| Anti-CD44 antibody | plasma membrane | Novus (47386) |
| Anti-N-cadherin antibody | plasma membrane | Abcam (ab18203) |
| Anti-ADAM22 antibody | plasma membrane | Stressmarq (SMC-412) |
| Anti-LRP4 antibody | plasma membrane | Stressmarq (SMC-418) |
| Anti-Fas antibody | plasma membrane | #12-0951-81 |
| (APO-1, CD95) | | (ThermoFisher) |
| Anti-CD27 antibody | plasma membrane | Abcam (ab70103) |
| Anti-EGFR antibody | plasma membrane | #MA5-13319 |
| | | (ThermoFisher) |
| Anti-Nesprin 2 antibody | nuclear envelope | #MA5-18075 |
| | | (ThermoFisher) |
| Anti-Nesprin 3 antibody | nuclear envelope | Novus (57690) |
| Anti-SYNE1 antibody | nuclear envelope | Prestige antibodies |
| | | (HPA019113) |
| Anti-SYNE2 antibody | nuclear envelope | Prestige antibodies |
| | | (HPA050204) |
| Anti-NUP214 antibody | nuclear envelope | Abcam (ab84357) |
| Anti-NPC antibody | nuclear envelope | SantaCruz sc-58815 |
| Anti-NUP88 antibody | nuclear envelope | Abcam (ab79785) |
| Anti-NUP98 antibody | nuclear envelope | Abcam (ab124980) |
| Biotinylated lectin WGA | nuclear envelope | B-1025 (VectorLabs) |
| (Wheat germ agglutinin) | | |

Preferably, the superparamagnetic beads have a size comprised between 100 nm and 100 μm, preferably between 100 nm and 50 μm, preferentially between 100 nm and 1 μm.

The size of the beads and their coating are chosen according to the intended use, taking into account the element, cell compartment, cell or set of cells targeted.

According to a particular embodiment, the beads used are chosen from:
(i) 800-nm beads, in particular streptavidin-coated beads, with non-specific aggregation and bonding properties to different biological structures such as the nucleus, the mitotic spindle centrosomes or the surface of certain cells.
(ii) 100-nm streptavidin-coated beads that do not exhibit specific interactions with the cell structures used for calibration and control experiments.
(iii) 200-nm magnetic beads including a fluorescent layer of the quantum dots type covered by purified molecular motors (dyneins), allowing, in particular, a more precise targeting to certain intracellular structures.
(iv) bovine serum albumin (BSA) passivated beads with a diameter of 1 μm, allowing, in particular, an inert displacement in the cytoplasm of the cells.
(v) 30-50-μm beads, in particular covered with a layer of agarose, which can especially be used to deform large portions of tissue, for example within the heart of an embryo.
(vi) 1-μm diameter beads (Dynabeads) associated with an anti-nuclear pore antibody (anti-NPC) allowing targeting to the nuclear envelope and deformation of the nucleus.

Preferably, the superparamagnetic particles used are fluorescent beads. Thus, the superparamagnetic beads according to the invention may also comprise one or more fluorescent molecules, such as a fluorophore, allowing their visualization by microscopy.

Thus, optionally, for the non-fluorescent particles, incubation with a biotinylated fluorophore (Atto-biotin, Sigma) can be carried out if visualization of the particles by fluorescence microscopy is necessary.

### Magnetic Tip

The present invention proposes the use of a magnetic tip for applying a force to the cell element or cell compartment of interest, by applying a magnetic field to the superparamagnetic beads.

‘Magnetic tip’ means a tip comprising a material which is either magnetizable or magnetized. The operation of the magnetic tip is based on a magnetic field gradient created by a tip comprising at least one magnet or electromagnet, intended to create a force on one or more magnetic beads attached to specific structures within or on the surface of cells.

Preferably, the magnet used is a permanent magnet. The term ‘permanent magnet’ is understood to mean a hard magnetic material whose remanent magnetization and coercive field are great. This type of magnet makes it possible to exert an attractive force on any ferromagnetic material. A magnet has a north pole and a south pole. Poles of the same nature repel, those of different natures attract.

According to one embodiment, the magnetic tips are composed of a magnetic core made of a superposition of millimetre or submillimetre sized magnets, whose geometry and magnetization can be modulated.

In particular, the magnetic tip comprises a magnetic core composed of at least 1, 2, 3 or 4 superimposed magnets, preferably 3 superimposed magnets.

Preferably, the magnet or magnets form the magnetic core and have a diameter comprised between 1 and 5 millimetres, preferably between 2 and 5 millimetres, between 3 and 4 millimetres, in particular 4 millimetres.

Preferably, the magnetic core has a cylindrical tubular shape. In particular, the diameter of the magnetic core is comprised between 1 and 5 millimetres, preferably between 2 and 5 millimetres, between 3 and 4 millimetres, between 2 and 3 millimetres, between 1 and 2 millimetres, between 1 and 3 millimetres, between 3 and 5 millimetres or between 4 and 5 millimetres. The length of the magnetic core is typically between 2 and 3 centimetres.

The magnet of the magnetic core is preferably made of neodymium, ticonal, alnico, neodymium iron boron (NdFeB) or samarium cobalt (SmCo). Preferably, the magnets of the magnetic core are made of neodymium.

Neodymium magnets can especially be coated with a thin layer of nickel-copper-nickel to protect them against corrosion.

The magnetic core is extended at its proximal end by a metal tip having a radius of curvature comprised between 5-100 micrometres, preferably between 10 and 90 micrometres, 20 and 70 micrometres, 5 and 50 micrometres, 10 and 50 micrometres or 25 and 50 micrometres. Preferably, the metal tip has a radius of curvature comprised between 5 and 50 micrometres.

According to one aspect, the metal tip has a radius of curvature adapted to the size of the biological sample and/or the element and/or the cell compartment and/or the mechanobiological property of interest.

According to one aspect, the length of the metal tip is between 1 and 30 millimetres, more particularly between 10 and 20 millimetres. The term ‘length’ is understood to mean the largest dimension of the metal tip, which extends along a longitudinal axis of said tip.

According to one aspect, the magnetic core, the length of the tip and the radius of curvature of the tip are adapted to allow a force to be applied to the biological sample in the range of 10 nN to 1000 nN.

In particular, the force applied to the biological sample is between 0.001 pN and 5000 nN, between 0.001 pN and 1000 nN, between 0.001 pN and 20 nN, between 0.001 nN and 5000 nN, between 0.01 nN and 1000 nN, between 0.001 nN and 20 nN or between 10 nN and 1000 nN, preferably between 0.001 pN and 100 nN, preferably between 0.5 pN and 1.5 nN.

The magnetic tip is adapted according to the force to be applied to the sample. Indeed, the force applied to a super paramagnetic bead of volume V with magnetic susceptibility χ for a field B is: F=Vχ*Grad(B²/(2μ₀)), where μ₀ is the susceptibility of the vacuum. The gradient function is a vector derivative that depends on the geometry of the tip and the position of the bead relative to the tip axis. For a bead exactly in the axis the grad is proportional to ˜ 1/L and 1/R where L is the length of the tip, and R is the radius of curvature.

The optimization of the length and curvature of the tip allows higher forces to be obtained despite the far working distance, and thus working on larger and/or more distant samples, which has never been achieved in the state of the art.

This tip makes it possible to channel the magnetic field lines on the beads, and thus to focus a greater magnetic field intensity on a smaller region of space.

In particular, the curvature of the magnetic tip is configured to create a uniaxial magnetic gradient on the biological sample.

The magnetic force applied by means of the curvature of the confined tip with a width of a few tens of micrometres allows the calibration of the system by applying a focused force in one direction, and thus ensuring that mechanobiological parameters can be measured accurately and reproducibly.

Preferably, the metal tip has a cylindrical tubular shape. The diameter of the metal tip may in particular be between 0.5 and 1 millimetre. The thinner tips provide finer spatial accuracy, and the wider tips provide a wider range of forces.

According to one embodiment, the metal tip may have a tapered or bevelled proximal end to facilitate passage of the magnetic tip through tissue.

‘Proximal end’ means the portion of the magnetic tip closest to the beads and/or the cell, tissue, cell element or compartment of interest. Conversely, the term ‘distal end’ is understood to mean the portion of the magnetic tip furthest from these elements.

Preferably, the metal tip overhanging the magnetic core is made of steel, iron, nickel or cobalt or one of their alloys, preferably steel.

According to one embodiment, the metal tip overhanging the magnetic core is covered with a layer of gold. In particular, the gold layer is applied by electronic sputtering to improve the stability of the tip with respect to oxidation in culture media, making it possible, in particular, to abolish toxic effects on the cells, and to preserve the tips for periods of use of several months/years.

According to a particular embodiment, the magnetic tip comprises a magnetic core composed of three neodymium magnets extended by a sharp steel tip having a radius of curvature of about 50 μm, capable of creating a magnetic gradient. The surface of the steel tip may in particular be covered with a layer of gold by electrolysis in order to prevent oxidation.

The magnetic tips are preferably calibrated by tracking the velocity of each of the magnetic beads in a medium of known viscosity as a function of the distance to the magnetic tip, and using Stokes' law to calculate the net magnetic force. The same experiment carried out on aggregates of beads of different sizes at a given distance made it possible to establish force-size relationships. The drag of the bead aggregates can be tested by measuring the sedimentation rate in a medium of known viscosity, and is represented by the drag of a spherical particle of radius equal to the geometric mean of the size of the aggregate (Tanimoto et al., Nature Physics 2018). One method of calibrating the tip and the beads is described in particular below.

The resulting force of the application of the magnetic tip on the beads depends in particular on three factors: (i) the magnetic properties of the tip and its curvature (ii) the magnetic susceptibility of the particles and their volume and/or (iii) the distance between the tip and the magnetic particle.

Method and Use

The invention particularly concerns a method for studying cellular and intracellular mechanics, especially within a biological sample, in particular a multicellular tissue and/or embryo, preferentially a non-human embryo.

The method according to the invention comprises a step of injecting the superparamagnetic beads, the injection being carried out using commercial injection systems (for example Eppendorf InjectMan) of magnetic particles or beads either within the cytoplasm of the cell, at the surface of the cell or within the lumen of a tissue or embryo. For example, using a 3D micromanipulator, the magnetic tip is approached at a controlled distance from the cell and the superparamagnetic beads, so that a precise force can be applied to the structure of interest. This application of force can be repeated several times during a cell cycle, maintained, or modulated by changing the position of the magnetic tip.

More particularly, the invention concerns an in vitro or ex vivo method for characterizing at least one mechanobiological property of at least one element, at least one cell compartment, at least one cell or at least one group of cells in a biological sample, said method comprising:
a) introducing the superparamagnetic beads into the biological sample, said beads comprising a coating capable of targeting the superparamagnetic beads to a cell element or compartment of interest or of penetrating a cell or group of cells,
b) applying a magnetic field to the superparamagnetic beads in order to apply a force to the biological sample,
c) measuring at least one mechanobiological property of the cell element or compartment in the biological sample,
   said biological sample being a cell or a multicellular biological sample, and
   the magnetic field being applied by a magnetic tip, said magnetic tip comprising at least one millimetre or submillimetre sized magnet extended by a metal tip.

According to one aspect, the coating is a hydrophobic coating and, preferably, the superparamagnetic beads comprising a hydrophobic coating are mixed with a biocompatible oil capable of causing the superparamagnetic beads to penetrate into a cell or group of cells, before being introduced into the biological sample.

According to another aspect, the coating is a biological coating capable of specifically targeting the superparamagnetic beads to a cell element or compartment of interest, a cell or a group of cells and preferably the superparamagnetic beads comprising a biological coating are mixed with a solution, preferably a buffer solution before being introduced into the biological sample.

The invention also concerns a method for characterizing one or more mechanobiological properties of a cell or one of its elements or compartments within a biological sample, said method comprising or consisting essentially of:
a) introducing superparamagnetic beads into the biological sample, said beads comprising a coating such as a biological coating capable of specifically targeting the superparamagnetic beads to a cell element or compartment of interest,
b) applying a magnetic field to the superparamagnetic beads in order to apply a force to the biological sample,
c) measuring one or more mechanobiological properties of the cell element or compartment in the biological sample.

The method can particularly be carried out in vivo, ex vivo or in vitro.

In particular, the invention concerns an in vitro or ex vivo method for characterizing at least one mechanobiological property of at least one element, at least one cell compartment, at least one cell or at least one group of cells in a biological sample, said method comprising:
a) introducing the superparamagnetic beads into the biological sample, said beads comprising a coating capable of specifically targeting the superparamagnetic beads to a cell element or compartment of interest,
b) applying a magnetic field to the superparamagnetic beads in order to apply a force to the biological sample,
c) measuring at least one mechanobiological property of the cell element or compartment in the biological sample, characterized in that
   said biological sample is a cell, a multicellular tissue or embryo, an organ, and said biological sample is not of human embryonic origin
   the magnetic field is applied by a magnetic tip, said magnetic tip comprising at least one millimetre or submillimetre sized magnet extended by a metal tip.

According to one aspect, the method further comprises (i) a preliminary step in which the biological coating of the beads is selected according to the cell element or compartment of interest, (ii) a preliminary step of preparing the tip and/or superparamagnetic beads and/or (iii) a step of calibrating the tip and beads.

According to another aspect, the method further comprises (i) a preliminary step of preparing the tip and/or superparamagnetic beads, (ii) a preliminary step in which superparamagnetic beads comprising the hydrophobic coating are mixed with a biocompatible oil, and/or (iii) a step of calibrating the tip and the beads.

The various steps of the method according to the invention are more particularly described below.

Biological Sample and Mechanobiological Characteristics Studied

The sample to which the method is applied may be an organism, embryo, organ, tissue or cell. The sample to which the method is applied is preferably selected from a cell, a multicellular sample, a tissue or an organ previously obtained by sampling or a preferably non-human embryo.

In particular, the biological sample is not a biological sample of human embryonic origin.

Preferably, the sample is in living and/or unfixed form.

According to one aspect, the sample is multicellular and is, in particular, a tissue, an organ or a preferably non-human embryo. The tissue can be of natural or synthetic origin, in two or three dimensions. In particular, the tissue may be a layer of cells, for example in cell culture.

According to a particular aspect, superparamagnetic beads are injected into the lumen of a tissue or organ. For example, it is possible to inject large magnetic beads into the lumen of the heart tissue of a vertebrate model embryo (zebrafish).

Preferably, the targeted cell element or compartment is the internal or external cell membrane of the biological sample.

The sample can be pre-treated, for example using a 50% bleach solution. The sample can also be slightly dried and then affixed to a glass slide and immediately covered with a drop of oxygen-permeable oil (for example with Voltalef Oil 10S).

The sample tested may be from a subject or from a cell culture, including using commercially available cell lines.

The sample may be, in particular, a biopsy, carried out on a subject prior to the method according to the invention. The terms ‘individual’, ‘host’, ‘subject’ and ‘patient’ are used here interchangeably, and denote a mammal, more particularly an animal subject and more particularly still a human. In the case of an animal, the animal may be a domestic animal such as a cat or dog, a farm animal such as a horse or cow, or a laboratory animal such as a mouse, a fruit fly, etc.

According to one embodiment, the method according to the invention is carried out on a cell. Preferably, the studied cell is a single cell or an isolated cell, a cell comprised in a pluricellular or multicellular tissue, a cell comprised in an embryo, said embryo preferably being non-human or a cell comprised in a biological sample, such as a biopsy or an explant. The cell may otherwise be a microorganism, such as a bacterium, yeast, or unicellular fungus.

In particular, the cell studied is a human, animal or plant cell. According to one embodiment, the cell is a non-human cell.

According to a particular embodiment, the cell is a tumour or cancerous cell. The cell may be from a primary tumour, a circulating cancer cell or a metastasis.

According to a particular embodiment, the sample on which the method according to the invention is carried out is an embryo, preferably a non-human embryo.

In particular, the embryo is chosen from a sea urchin (Paracentrotus lividus), fruit fly (Drosophila melanogaster) or zebrafish (Danio rerio) embryo.

Preferably, the cell compartment or element targeted by the superparamagnetic beads is chosen from: the cell membrane, nuclear membrane, membrane of an organelle such as mitochondria, endoplasmic reticulum or Golgi apparatus, peroxisomes, lysosomes and their contents, cytoplasm, microtubules and/or centrioles.

Preferably, the targeted cell compartment or element is the cell membrane or nuclear membrane.

The method according to the invention makes it possible to evaluate the mechanobiological characteristics of these different elements.

The terms ‘mechanobiological characteristics’ and ‘mechanobiological properties’ are used interchangeably here and relate to the effects of mechanical stresses (stretching, compression, shearing etc.) on living cells or tissues. Mechanobiology encompasses rheology, kinematics, mechanical strength, elasticity, plasticity, stiffness, flexibility and viscoelastic properties, or any combination thereof.

According to one embodiment, the mechanobiological properties are chosen from: rheology, kinematics, mechanical strength, elasticity, plasticity, stiffness, flexibility and viscoelastic properties, or any combination thereof. Preferably, the mechanobiological property measured is stiffness or elasticity.

‘Rheology’ is the science that studies the deformations and flow of matter. Its purpose is to analyze the mechanical behavior of substances and to establish their behavior laws. It encompasses many fundamental disciplines such as material strength, fluid mechanics, plasticity and elasticity. The rheological properties of a material or cellular element correspond to the mechanical behavior and the relationships between the deformations and stresses of the material. The rheology of the cytoplasmic fluid of a cell or the lumen of a tissue can be measured by the proposed magnetic tip method. For this purpose, beads of different sizes can be displaced by the application of a magnetic force, making it possible to determine the viscosity of the cytoplasm, the dependence of this viscosity according to the size of the object displaced or the shear force. Typical viscosities of the cytoplasm range from 10-1000 cP, and depend on the size of the object displaced in the fluid as well as the cell type.

The ‘mechanical strength’ of an element is its global behavior (relationship between stresses−forces or moments—and displacements) and its local behavior (relationship between stresses and deformations). When the intensity of the stress increases, there is first elastic deformation (the element deforms proportionally to the force applied and resumes its initial shape when the stress disappears), followed sometimes by plastic deformation (the element does not resume its initial shape when the stress disappears, there remains residual deformation), and finally rupture (the stress exceeds the intrinsic strength of the material). The mechanical strength of the cell or nuclear membrane or of a tissue layer, can be probed by applying the magnetic tips according to the invention. In particular, the precise application of surface forces, and the concomitant measurement of the deformation, makes it possible to determine the stress-strain relationships of these biological surfaces, and to calculate, for example, elasticity constants ranging from 10 to 1000 pN/μm, Young's moduli of around 1-100 MPa or breaking point deformations for these surfaces of 5-50%.

‘Viscoelasticity’ is the property of materials that exhibit both viscous and elastic characteristics when subjected to deformation. Elastic materials deform when stressed, and quickly return to their original state when the stress is removed. In rheology, the behavior of a linear viscoelastic material is thus intermediate between that of an ideal elastic solid symbolized by a spring of modulus E (or G) and that of a Newtonian viscous liquid symbolized by a damper of viscosity η. The elasticity of a material reflects its ability to conserve and restore energy after deformation. The viscosity of a material reflects its ability to dissipate energy. The viscoelastic properties of the cell or nuclear membrane, or of a tissue layer can be tested by applying the magnetic tips according to the invention. In particular, the precise application of surface forces and the dynamic measurement of the resulting deformation make it possible to characterize the viscoelastic timescales, and the associated viscous and elastic constants. Some materials can especially exhibit short-timescale elastic properties and long-timescale viscous properties (Maxwell materials) or conversely they flow in short timescales and behave like an elastic solid in long timescales (Kelvin-Voigt materials). The viscoelastic timescales for the biological materials considered by the invention can vary between 0.1-1000 s; the viscous friction constants between 100-10,000 pN/μm, and the elastic constants from 10 to 1000 pN/μm. ‘Rigidity’ or ‘stiffness’ is the characteristic that indicates the resistance to elastic deformation of a body. The more rigid an element is, the greater the force applied to it must be in order to obtain a given deformation. Conversely, ‘flexibility’ refers to the property that a flexible material can be easily curved or bent without breaking.

The ‘mechanical strength’ of a material is characterized by its elastic limit and/or tensile strength.

The mechanobiological properties can in particular be evaluated by measuring the Young's modulus. An element with a high Young's modulus (or modulus of elasticity) (>50 GPa) is said to be rigid, the element with a low Young's modulus is, on the contrary, soft, elastic or flexible.

Preparation of the Magnetic Tip and Paramagnetic Beads

According to one embodiment, the method according to the invention may also comprise a step of preparing the tip and/or the paramagnetic beads.

According to one embodiment, the method according to the invention further comprises the preparation or manufacture of a tip comprising or consisting in the assembly of elements necessary and appropriate for its magnetic use.

The manufacture of the tip consists mainly of the assembly of a magnetic core and a metal tip as described above in the ‘metal tip’ section.

Thus, the characteristics of the tip can be optimized according to the sample to be evaluated.

According to one embodiment, the magnetic tip is a magnetic tweezer.

According to one embodiment, the method according to the invention also comprises the choice and the preparation of the superparamagnetic beads prior to their introduction into the cell, tissue, organ or embryo.

The targeting of magnetic particles to a specific subcellular location can be controlled by adding “hook” proteins as a biological coating on the surface of the particles, or beads. In most cases, these proteins are biotinylated for efficient coupling with the streptavidin group present on the surface of the magnetic particles.

In practice, in a first step, the particles are washed in a saline solution or a buffer solution, for example phosphate buffer (PBS). The particles are then incubated in a solution containing the hook protein. For example, for targeting the nuclear envelope, a biotinylated antibody directed against nuclear pores such as anti-NPC antibody (SantaCruz sc-58815) may be used.

Preferably, excess hook protein is removed by washing in a buffer solution, for example PBS.

The functionalized particles are finally re-suspended in PBS at a concentration suitable for injection.

Preferably, the concentration of the functionalized paramagnetic beads is between 0.5 and 5 mg/mL in the injection solution.

For antibodies that are not available in biotinylated version, they can be biotinylated, in particular by the prior use of a biotinylation kit (e.g.: EZ-link NHS-biotin, Sigma).

Optionally, when the beads used are non-fluorescent, a second incubation with a biotinylated fluorophore, for example the Atto-biotin fluorophore (Sigma) can be carried out at this stage if visualization of the particles by fluorescence microscopy is necessary.

Preferably, excess fluorophore is removed by washing in a buffer solution, for example PBS.

The functionalized particles are finally re-suspended in PBS at a concentration suitable for injection.

Preferably, the concentration of the functionalized paramagnetic beads is between 0.5 and 5 mg/mL in the injection solution.

It is possible to verify the functionalization of the beads by dynamic light scattering (DLS), which makes it possible to measure the size distribution of objects suspended in a solution.

Thus, it is possible to compare the size of the initial beads and the size of the functionalized beads, the functionalized beads having a size greater than the non-functionalized beads.

According to one embodiment, the method according to the invention also comprises a step of verifying the functionalization of the beads before injection, for example by the DLS technique.

In the case of superparamagnetic beads comprising a hydrophobic coating, commercial beads are available such as, for example, Magtivio, MagSi-proteomics C18 beads, which provide a coating made of carbon chains of the C18 octadecyl type. For injection, the beads are dried with ethanol and then re-suspended in biocompatible oil, preferably soya oil, at a concentration comprised between 5 and 20 mg/mL.

To inject the oil droplets comprising the superparamagnetic beads comprising a hydrophobic coating into the cytoplasm, a suspension of hydrophobic superparamagnetic beads is washed in a 30, 50 and then 70% ethanol solution. This solution is then dried under vacuum and resuspended in the biocompatible oil.

Preferably, the concentration of the beads comprising a hydrophobic coating is between 2 and 20 mg/mL, preferably between 5 and 20 mg/mL in the injection solution, i.e., in the biocompatible oil.

Calibration

To know the precise force applied to a bead by the presence of the magnetic tip and thus evaluate the mechanobiological properties of the cell element or compartment, it may be necessary to calibrate the tip-bead pair.

Thus, the method according to the invention may further comprise a method for calibrating the magnetic tip-superparamagnetic beads pair, preferably prior to testing the sample of interest.

This calibration can be carried out according to a protocol consisting of placing the beads in a solution of known viscosity. When the magnetic tip is placed in the solution, the beads move at a speed that allows calculating the Stokes viscous friction force (F=6πηRV, η: viscosity of the liquid, R radius of the bead, V velocity of the bead) that balances the magnetic force. This method involves the use of liquids of calibrated viscosity, such as dilute solutions of sucrose (52%, 12 cP; 54%, 20 cP), glycerol (68%, 15 cP; 71%; 20 cP, 80%, 80 cP), methyl cellulose 15 (2%, 15 cP), or methyl cellulose 400 (1.18%, 39 cP; 1.60%, 124 cP; 2%, 400 cP). The diluted magnetic beads are suspended in a flux chamber on a microscope. The beads are then set in motion by the approach of the magnetic tip in the solution, and the distance between the tip and the bead, as well as the speed of the bead, are recorded by videomicroscopy. Image analysis scripts established in the context of this invention make it possible to extract the important parameters (distances and velocities) and thus give the value of the force as a function of the position and axis of the bead with respect to the tip. For the same tip, this calibration can be carried out for different types of beads. Thus, once the calibration has been performed, the script automatically extracts the value and direction of the force during an experiment on living samples.

With this system, the force can be modulated by changing the position of the magnetic tip in the case of a permanent magnet or by changing the electric intensity in the case of an electromagnet.

Introduction of Magnetic Beads

The method according to the invention comprises a step comprising or consisting in introducing superparamagnetic beads into a cell, tissue, organ or embryo, especially as described above, said beads comprising a coating enabling the superparamagnetic beads to be specifically targeted to a given element or compartment or to penetrate a cell or group of cells.

This step can be based, in particular, on the use of conventional microinjectors, available in biology laboratories, such as 3D microinjectors.

To inject beads into a cell or tissue, the items to be optimized include, for example, the size of the microcapillary opening, injection pressure, compensation pressure and injection duration. For example, the in vivo injection of a solution of magnetic beads from 800 nm to 5 mg/mL is performed with the following parameters: Capillary opening: 5 to 15 micrometres, compensation pressure: 5 to 30 hectopascals, injection pressure: 15 to 90 hectopascals and duration of injection: 100 to 500 milliseconds. These protocols have been refined within the scope of this invention.

For cell surface measurements, beads comprising a biological coating are most often targeted by adding magnetic particles to the medium and allowing them to adhere to the external cell surface.

According to one embodiment, the beads are introduced into the biological sample by microinjection, by a cellular transport route (such as cellular endocytosis), by permeabilization of the membrane of the biological sample of interest, by biolistics or by bombardment, preferably by microinjection, preferentially by pneumatic or hydraulic microinjection.

In particular, either of two pneumatic injection systems can be used: FemtoJet4 (Eppendorf) and PicoPump PV820 (WPI). Both systems have the advantage of being able to precisely control the injection and compensation pressure as well as the injection duration.

Alternatively, the CellTram4r Oil hydraulic system (Eppendorf) makes it possible to work comfortably in higher pressure ranges (>500 hPa), especially by reducing inertia during injection.

The choice between the systems is made according to the type of sample, the type of needle and the type of suspension to be injected. For example, the hydraulic injection system can be used for experiments in multicellular samples and, more particularly, in non-human embryos, such as fruit fly embryos.

According to one embodiment, the beads are injected into the cell by biolistics or bombardment. This technique consists in propelling the superparamagnetic beads of interest into the cells using a particle gun (for example Biolostic—Biorad PDS-1000/He).

According to one embodiment, the beads are introduced into the sample of interest by the introduction of an injection needle.

Injection needles are preferably prepared from borosilicate glass capillaries using a stretcher, for example the P-1000 stretcher from Sutter Instrument. The capillaries can be treated beforehand with a siliconizing solution, for example Sigma's Sigmacote solution.

Preferably, the needles have a diameter comprised between 1 μm and 1 mm and are particularly adapted to the diameter of the beads to be injected. The needles are preferably sharpened at an angle of 100 to 300+/−2°, preferably about 300 until an opening is obtained, for example with the aid of a beveler (EG-40, Narishige). The diameter of the opening of the injection needle may be between 1 μm and 1 mm, preferably between 5 and 15 micrometres.

Preferably 1 to 5 microlitres of injection solution comprising the superparamagnetic beads in concentrations of preferably 0.5 to 5 mg/mL for beads comprising the biological coating in the injection solution, preferably in phosphate buffer, and 5 to 20 mg/mL for beads with hydrophobic coating in oil, are introduced, at the back of the needle, just before mounting on the injection system.

According to one aspect, several beads are injected during step a) of the method according to the invention into at least one cellular compartment of the biological sample.

According to another aspect, the superparamagnetic beads are introduced into a single cell of the biological sample.

Application and Measurement of Forces in Living Samples.

The method comprises a step comprising or consisting in applying a magnetic field to said superparamagnetic beads, so as to apply a force to the biological sample. This makes it possible to evaluate and/or measure the mechanobiological properties of the cell element or compartment in the biological sample.

The magnetic tip system is compatible with different types of microscopy, allowing visualization of the application of the magnetic tip near the beads and the displacement of the superparamagnetic beads.

The tissue or cell in question is imaged on an inverted or upright microscopy system, and after injection or positioning of the magnetic beads, the magnetic tip is brought into the vicinity of the sample to apply a force and a magnetic field. The position and/or orientation of the tip is controlled by means of a micromanipulator, for example a commercial 3D micromanipulator. The microscopy images taken must contain the tip and the magnetic particle(s) and make it possible, by means of computer scripts, to calculate the exact force as a function of the particle size, the distance between the magnetic tip and the beads, and the calibration mentioned above. Depending on the process, monitoring the deformation of the tissue, cellular or nuclear surface, or of the movement of an organelle by videomicroscopy ultimately makes it possible to calculate the mechanobiological properties of the studied element (elastic stiffness constant, breaking point, viscous friction, or viscoelastic timescale).

In particular, the distance between the magnetic tip and the biological sample is between 10 micrometres and 1 millimetre.

According to one embodiment, the microscope used is a wide field fluorescent microscope or a confocal microscope. The microscope can be equipped with a camera. The microscope can also be controlled by Micro-Manager (Open Imaging).

Preferably, images are taken at room temperature (18-20° C.).

In order to obtain the most robust information possible, the force applied to the cell via the magnetic tip and the superparamagnetic beads must be constant and stable for an instant t. The position of the tip must thus be maintained in a stable manner close to the beads.

According to one embodiment, the tip is positioned between 10 and 500 μm from the cell or cell element or compartment of interest. According to one embodiment, the tip is positioned between 10 and 500 μm away from the surface of the superparamagnetic beads.

Preferably, the distance between the tip and the multicellular sample is between 10 micrometres and 1 millimetre. This distance can be adapted according to the size of the biological sample and the force to be applied to it.

According to one aspect, the force applied to the biological sample is between 0.001 pN and 5000 nN, between 0.001 pN and 1000 nN, between 0.001 pN and 200 nN, between 0.001 pN and 20 nN, between 0.001 nN and 5000 nN, between 0.01 nN and 1000 nN, between 0.001 nN and 200 nN, between 0.001 nN and 20 nN, between 0.1 nN and 200 nN, between 0.1 nN and 20 nN, between 0.5 nN and 200 nN, between 0.5 nN and 20 nN, between 0.5 nN and 10 nN, between 0.5 nN and 5 nN, between 1 nN and 200 nN, between 1 nN and 20 nN, between 1 nN and 5 nN, between 5 nN and 50 nN, or between 10 nN and 1000 nN, between 50 nN and 500 nN, 0.001 pN and 100 nN, preferably between 0.5 pN and 1.5 nN, most preferably between 0.5 nN and 1.5 nN.

In particular, the force applied to the biological sample is between 0.001 pN and 1000 nN, preferably between 0.001 pN and 200 nN, preferentially between 0.5 pN and 1.5 nN. Preferably, the force applied to the biological sample is between 10 nN and 1000 nN.

Scope of the Method

The use of superparamagnetic beads and the magnetic tip allows exploring the impact of the mechanical environment (stimulus) on cells, studying the perception of stimulus and the response mechanisms of cells and their participation in mechanical and biological processes.

According to a particular aspect, the method relates to a multicellular biological sample and makes it possible to characterize at least one cell compartment, cell or group of cells.

According to another aspect, the method makes it possible to characterize the at least one element or the at least one cellular compartment of the multicellular biological sample.

The invention concerns the use of the method according to the invention for:
(i) characterizing the mechanobiological properties of a cell or one of its elements or compartments, a collection of cells and/or
(ii) characterizing the impact of a drug or medicament on a cell, tissue, organ or embryo or any of its elements or compartments and/or
(iii) identifying and characterizing damaged cells or tissues, e.g., damaged heart cells in heart or genetic diseases and/or
(iv) identifying and characterizing tumour cells in a sample, which tumour cells exhibit mechanobiological characteristics different from normal cells and/or
(vi) understanding the mechanical interaction of cells with each other or with the surrounding matrix that allows them to form coherent tissues and/or
(vii) studying mechanotransduction, i.e., how the cell integrates the mechanical signal and transforms it into a biochemical signal that allows it to react, adapt and even modify the expression of its genes. Mechanotransduction is involved in many cellular functions such as shape change, differentiation, growth, proliferation and apoptosis. Three types of mechanotransduction are currently described as a function of the transduction used by the cell to adapt its response to environmental factors. The first is the opening of ion channels following mechanical stress, the second is the initiation of a signaling cascade and the third takes a physical bridge between the focal points and chromatin.

Kit and Use of the Kit

Finally, the present invention relates to a kit for performing the method according to the invention.

According to one aspect, the kit includes:
a) a magnetic tip comprising a magnetic core comprising a superposition of millimetre or submillimetre sized magnets extended by a steel tip,
b) superparamagnetic beads comprising a coating capable of targeting the superparamagnetic beads to a cell element or compartment of interest or penetrating a cell or group of cells,
c) optionally, a solution allowing to suspend and inject the superparamagnetic beads into a cell or cell tissue and
d) optionally at least one solution for calibrating the superparamagnetic beads and the magnetic tip.

According to one aspect, the coating is a hydrophobic coating, preferably octadecyl, the solution being a biocompatible oil capable of causing the superparamagnetic beads to penetrate into a cell or group of cells.

According to another aspect, the superparamagnetic beads comprise a biological coating capable of specifically targeting the superparamagnetic beads to a cell element or compartment of interest, the coating of the superparamagnetic beads preferably being a protein coating, preferentially an antibody or a lectin, the solution being a buffer solution, preferably a phosphate buffer.

In particular, the kit includes:
a) a magnetic tip, preferably as described above in the ‘magnetic tip’ section,
b) superparamagnetic beads suitable for the specific targeting and attachment of said superparamagnetic beads to a given cell element or compartment, preferably as described above in the ‘superparamagnetic beads’ section,
c) optionally a solution allowing the functionalization of the superparamagnetic beads, for example comprising antibodies or a bait protein,
d) optionally, one or more solutions for the calibration of the beads and the magnetic tip,
e) optionally a solution for suspending and injecting superparamagnetic beads into a cell or cell tissue;
f) optionally, an instrument for injecting the beads, preferably a microinjector or injection needle prepared from borosilicate glass capillaries.

In particular, the kit comprises:
a) a magnetic tip, preferably as described above in the ‘magnetic tip’ section,
b) superparamagnetic beads comprising a hydrophobic coating, preferably as described above in the ‘superparamagnetic beads’ section,
d) optionally, one or more solutions for the calibration of the beads and the magnetic tip,
e) an oil for suspending and injecting superparamagnetic beads into a cell or cell tissue, preferably as described in the ‘superparamagnetic beads’ section;
f) optionally, an instrument for injecting the beads, preferably an injection needle prepared from a glass capillary or a microinjector.

The kits according to the invention may also comprise one or more consumables such as a culture medium or a preservation medium for the tissues or cells, or an explanatory leaflet.

Preferably, the invention relates to a kit for characterizing at least one mechanobiological property of a cell element or compartment in a biological sample comprising:
a) a magnetic tip comprising a magnetic core comprising a superposition of millimetre or submillimetre sized magnets extended by a steel tip
b) superparamagnetic beads comprising a biological coating capable of specifically targeting the superparamagnetic beads to a cell element or compartment of interest, the coating of the superparamagnetic beads preferably being a protein coating, preferentially an antibody or a lectin,
c) optionally, a solution for suspending and injecting the superparamagnetic beads into a cell or cell tissue and
d) optionally at least one solution for calibrating the superparamagnetic beads and the magnetic tip
and characterized in that said biological sample is a cell, multicellular, tissue, organ, embryo and said biological sample is not a biological sample of human embryonic origin.

Preferably, the kit comprises:
a) a magnetic tip comprising a magnetic core comprising a superimposition of millimetre or submillimetre sized neodymium magnets extended by a machined steel tip with a radius of curvature comprised between 5 and 100 micrometres,
b) superparamagnetic beads comprising a coating making it possible to specifically target the superparamagnetic beads to a given cell element or compartment, the coating of the superparamagnetic beads being a protein coating, preferably an antibody or a lectin,
c) optionally, a solution for suspending and injecting the superparamagnetic beads into a cell or cell tissue, and
d) optionally, an instrument for injecting the beads, preferably an injection needle prepared from borosilicate glass capillaries.

In particular, the magnetic core of the tip, the length of the tip and the radius of curvature of the tip shall be adapted so as to allow a force comprised between 10 nN and 1000 nN to be applied to the biological sample.

In particular, the steel tip has a length comprised between 1 and 30 mm and/or the radius of curvature of the steel tip is between 5 and 100 micrometres.

According to one aspect, the magnet of the magnetic core is made of neodymium, ticonal, alnico, neodymium iron boron (NdFeB) or samarium cobalt (SmCo), preferably neodymium.

Finally, the invention concerns the use of the kit as described above, for
(i) characterizing the mechanobiological properties of a cell or any of its elements or compartments, and/or
(ii) characterizing the impact of a drug or medicament on a cell, tissue, organ or embryo, in particular a non-human embryo, or any of its parts or compartments, and/or
(iii) identifying tumour cells in a sample, which tumour cells have mechanobiological characteristics different from normal cells,
(iv) identifying and characterizing damaged cells or tissues, and/or
(vi) studying the mechanical interaction of cells with each other or with the surrounding matrix, and/or
(vii) studying mechanotransduction, that is, how the cell integrates the mechanical signal and transforms it into a biochemical signal that allows it to react, adapt and even modify the expression of its genes.

### Examples

Materials and Methods

Fabrication of Magnetic Tips

The magnetic tips are composed of a magnetic core made of a superimposition of submillimetre or millimetre sized neodymium magnets, whose geometry and magnetization can be modulated. This core is extended by a machined steel tip with a radius of curvature that can range from 5-100 micrometres. Thinner tips provide finer spatial accuracy, and wider tips provide a greater range of forces. To stabilize the tips against oxidation in the culture media, and thus abolish the toxic effects on the cells, the tips are covered with a thin layer of gold, in particular with a thickness of 0.5 to 1 micrometre by electronic sputtering. The electronic sputtering parameters are to be adapted for each tip size and each metal alloy used in order to obtain a continuous gold layer. The tips may in particular be made of steel. Tips made in this way can be kept dust-free for several years.

Preparation and Selection of Magnetic Particles.

The method is compatible with all kinds of superparamagnetic particles, exhibiting a variable range of surface chemistry, especially making use of avidin-biotin bonds, or carboxyl group sulfonation reactions. The inventors tested and validated the method of the invention with a dozen different brands of beads, without any sign of defect or limit of the approach. The choice of particles and the modification of their surface chemistry allows a very wide field of applications.

The targeting of magnetic particles to a specific subcellular location can be controlled by adding hook proteins (with specific affinity to a compartment/structure) to the surface of the particles. In most cases, these proteins are biotinylated for efficient coupling with the streptavidin group present on the surface of the magnetic particles.

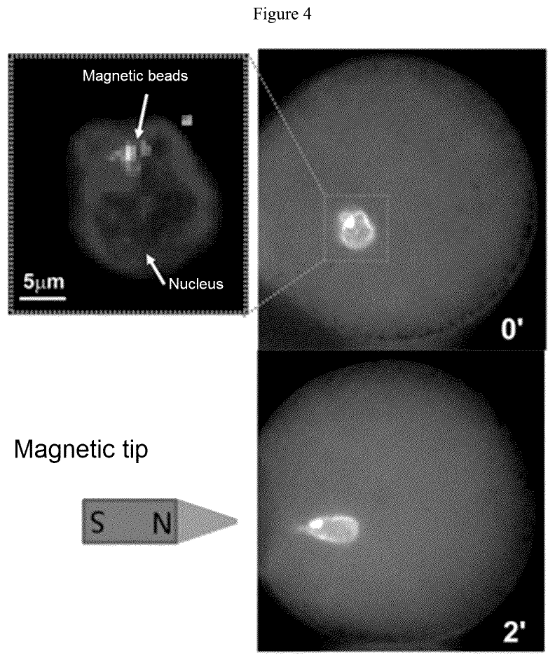

In practice, in a first step, the particles are washed in a solution containing 1 M NaCl, 1% Tween and then resuspended in PBS (phosphate buffer saline). If necessary, the particles are then incubated for 15 to 60 min in a solution containing the required hook protein. For example, for targeting the nuclear envelope, a biotinylated antibody directed against nuclear pores (mouse anti-NPC antibody—SantaCruz sc-58815)) was used and validated (FIG. 5). Excess protein is removed by washing in PBS. Optionally, for nonfluorescent particles, a second incubation with biotinylated fluorophores (Atto-biotin, Sigma) can be carried out at this stage if visualization of the particles by fluorescence microscopy is necessary. The particles are finally resuspended in PBS at a concentration comprised between 0.5 and 5 mg/mL. This injection solution can be used for several hours.

Examples of hook proteins that can be used to target particles to the plasma membrane or nuclear envelope are described in Table 1.

For antibodies not available in biotinylated versions, prior use of a biotinylation kit is required (e.g.: EZ-link NHS-biotin, Sigma).

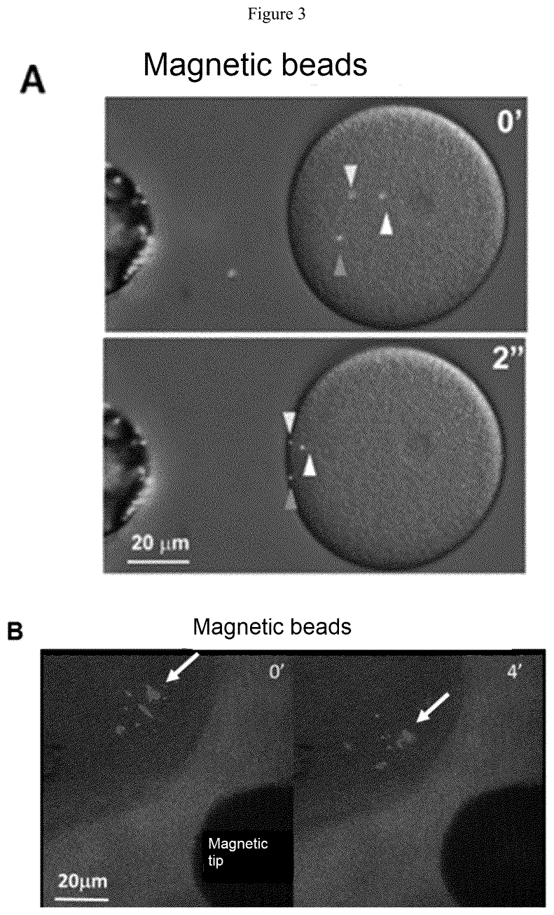

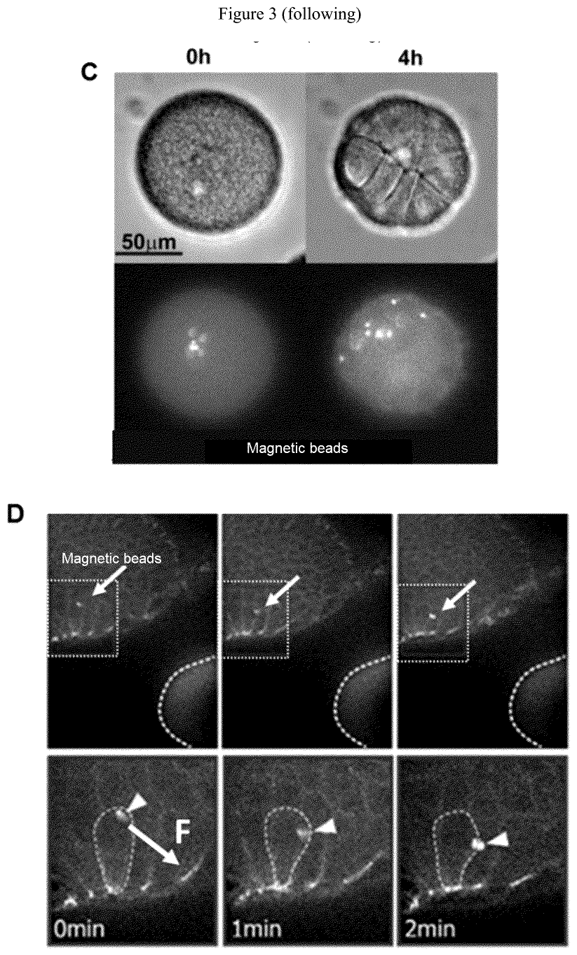

In particular, the inventors used and validated protocols using (i) 800-nm beads of the NanoLink brand (Solulink), which have nonspecific aggregation and bonding properties to different biological structures such as the nucleus, the mitotic spindle centrosomes or the surface of certain cells (FIG. 3A-3B). (ii) 100-nm streptavidin (Chemicell) coated beads that do not exhibit specific interactions with cell structures used for calibration and control experiments. (ii) 200-nm streptavidin magnetic quantum dots (Nvigen) covered by purified molecular motors (dyneins) allowing more precise targeting to certain intracellular structures. (iii) bovine serum albumin (BSA) passivated beads 1 μm in diameter (Dynabeads) allowing an inert displacement in the cytoplasm of the cells (FIG. 3C). (iv) 30-50-μm beads (Magne) to deform large portions of tissue within the heart of a zebrafish embryo (FIG. 3D). (v) 1-μm diameter beads (Dynabeads) associated with an anti-nuclear pore antibody (anti-NPC) allowing targeting to the nuclear envelope and deformation of the nucleus (FIG. 4).

Calibration of the Applied Force

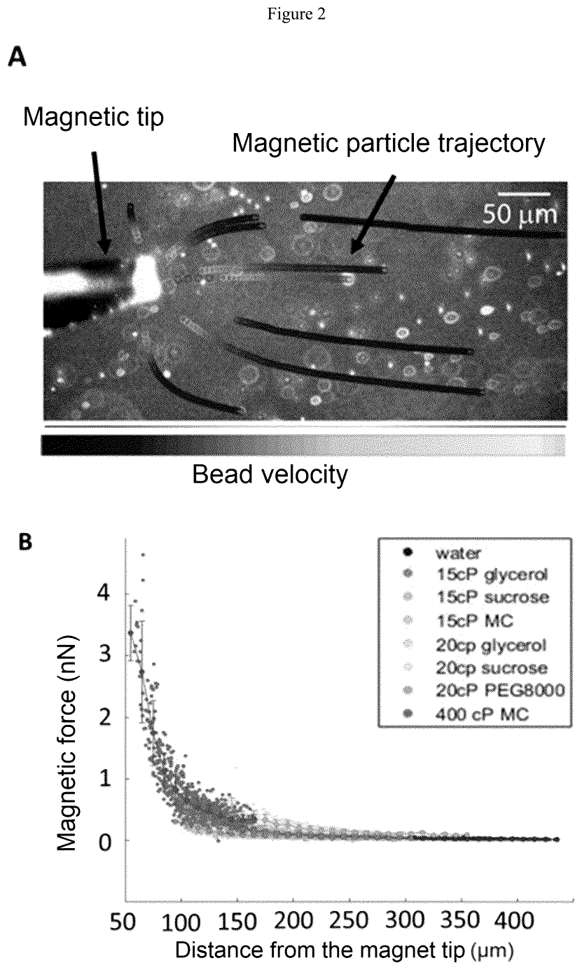

To know the precise force applied to a bead by the presence of the magnetic tip, it is necessary to calibrate the magnetic tip-bead pair (FIG. 2). This calibration makes it possible to know the exact force according to the distance between the bead and the magnetic tip. For this purpose, a suspension of dilute beads (1.25%) is placed in a solution of known viscosity. The viscous agents diluted in water that have been tested and validated are: Glycerol (Sigma) 60% viscosity 15 cP (or mPa·s), sucrose (Sigma) 65% viscosity 15 cP; methyl cellulose 15 2% (Fisher Scientific); viscosity 15 cP; glycerol 65%, viscosity, 20 cP; sucrose 80%, viscosity 20 cP; polyethylene glycol 8000 (Sigma), viscosity 20 cP; methyl cellulose 400 2% (Fisher Scientific), 400 cP (FIG. 2B). When the magnetic tip is placed in the solution, the beads move at a speed that allows calculating the Stokes viscous friction force (F=6πηRV, η: viscosity of the liquid, R radius of the bead, V velocity of the bead) that balances the magnetic force. An image analysis script developed in Matlab software (Mathworks), allows: (i) the dynamic tracking of the particle, and the calculation of its velocity, and (ii) the precise positioning of the center of the tip as a reference. This script thus directly extracts the magnetic force-distance relationship at the magnetic tip, as well as refinements of this force according to the angle between the trajectory of the bead and the main axis of rotation of the magnetic tip. For the same tip, this calibration can be carried out for different types of beads. Thus, once the calibration has been performed, the script automatically extracts the value and direction of the force during an experiment in living samples. This is described in particular in the publication by Tanimoto et al., Nature Physics 2018.

Magnetic Bead Injection

This step involves the use of conventional microinjectors available in biology laboratories. To inject beads into a cell or tissue, the items to be optimized include the size of the opening of the injection micro-capillary, the pressures and the duration of injection. For cell or tissue surface measurements, the methods are more direct and usually consist of adding the magnetic particles to the medium and allowing them to stick to the cell surface.

Injection needles are prepared from borosilicate glass capillaries (diameter 1 mm) using a stretcher (P-1000; Sutter Instrument). The capillaries are treated beforehand with a siliconization solution (Sigmacote, Sigma). The needles are sharpened at an angle of 300 to obtain an opening of 5-10 micrometres using a beveler (EG-40, Narishige). 2 to 3 microlitres of injection solution are introduced into the back of the needle just before mounting on the injection system.

The injection of large beads (20-60 μm) into the heart tissues of the zebrafish embryo is done by incising the tissues between the sinus and atrium and pushing the bead into the atrium using a needle placed on a micromanipulator under the microscope.

The sea urchin embryos are injected into a glass-bottomed chamber containing seawater. The glass is pretreated beforehand with a 1% protamine solution (Sigma) to ensure the adhesion of the sample. The injection needle is controlled by a motorized micromanipulator (InjectMan 4, Eppendorf) and an injection pump (FemtoJet, Eppendorf).

Fruit fly embryos are harvested 1 hour maximum after laying and dechorionated in a 50% bleach solution. They are then slightly dried and then affixed onto a glass slide. They are immediately covered with a drop of oxygen-permeable oil (Oil 10S Voltalef). The injection is performed with a hydraulic micromanipulator (Narishige) at the anterior end of the embryo before the cellularization phase. The injection pressure is controlled manually using a microinjector (CellTram, Eppendorf).

Injection of Beads Mixed with Oil

To measure the properties of the cytoplasm at the scale of large passive objects, magnetized oil droplets in the cytoplasm are used. For this, unfertilized sea urchin eggs are placed in cell culture Petri dishes with a glass bottom, previously covered with a protamine solution. To inject the magnetized oil droplets into the cytoplasm, a suspension of 10 μl of hydrophobic superparamagnetic beads with a diameter of 1.2 μm (Magtivio, MagSi-proteomics C18) is washed in 100 μl of 30, 50 and then 70% ethanol solution. This solution is then dried under vacuum for 20 minutes and resuspended in 5 μl of soya oil (soya oil, Naissance). Glass capillaries for injection are stretched with a capillary stretcher (P-1000, Sutter Instrument) and machined at an angle of 40° on a diamond wheel (EG-40, Narishige) to obtain a 10-μm opening. The injection capillaries are filled with 2 μL of oil solution allowing several cells to be injected and are not reused. The injection time defines the size of the drop formed in the cytoplasm. After injection, the magnetic tip is brought close to the cell and the beads inside the oil form aggregates, which initially move slowly towards the magnet while the oil droplet remains stationary. When the aggregate comes into contact with the oil-cytoplasm interface facing the magnet tip, the magnetic force is transmitted to the drop and displaces it within the cell. Large aggregates are more suitable for the method, as they generally cannot cross the interface due to the surface tension of the oil, and the hydrophobic properties of the beads. The force is calibrated by measuring the size of the aggregate obtained by videomicroscopy.

Cell and Tissue Culture

Adult sea urchins (Paracentrotus lividus) originate from the marine stations of Roscoff and Villefranche sur Mer (France) and are kept for several weeks in aquariums of artificial seawater maintained at 16° C. The gametes are obtained by intracoelomic injection of 0.5 M KCL. The eggs are kept in seawater at 16° C. for a maximum of 12 hours. The sperm can be stored undiluted for 1 week at 4° C. The egg matrix is removed by passing it through a mesh of 80 micrometres. Before each experiment, the sperm is activated by dilution and brought into contact with the eggs for fertilization.

The fruit flies (Drosophila melanogaster) are maintained at 25° C. in tubes containing a nutrient medium. For the collection of embryos, the fruit flies are transferred into laying pots containing an agar medium supplemented with fresh yeast. The laying pots are replaced every 30-60 minutes and the embryos are collected by rinsing the pots with water and separating the embryos from the yeast using a sieve. As the embryos are developing, they are prepared for injection immediately.

Zebrafish embryos (Danio rerio) are cultured in standard enriched medium (https://zfin.org/zf_info/zfbook/chapt1/1.3.html); for 2-3 days after fertilization at ˜28° C. and prepared for bead injection (see above). They are turned so that the heart is facing the coverslip under a binocular, then imaged directly with a microscope equipped with magnetic tips.

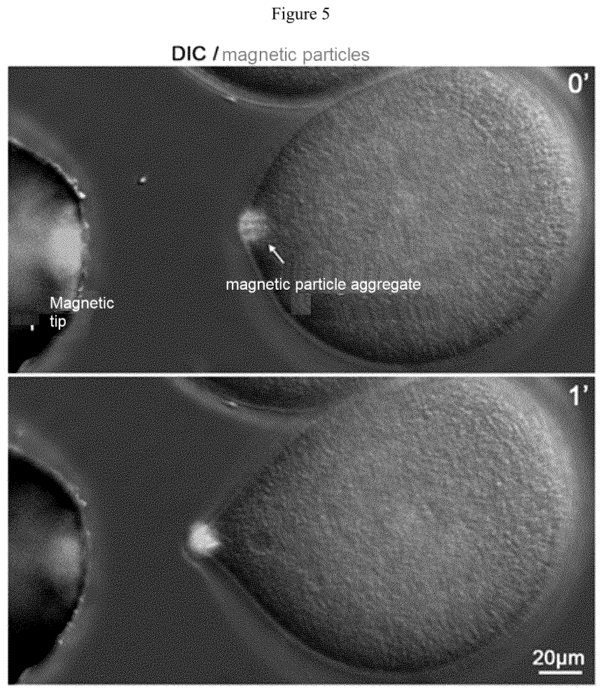

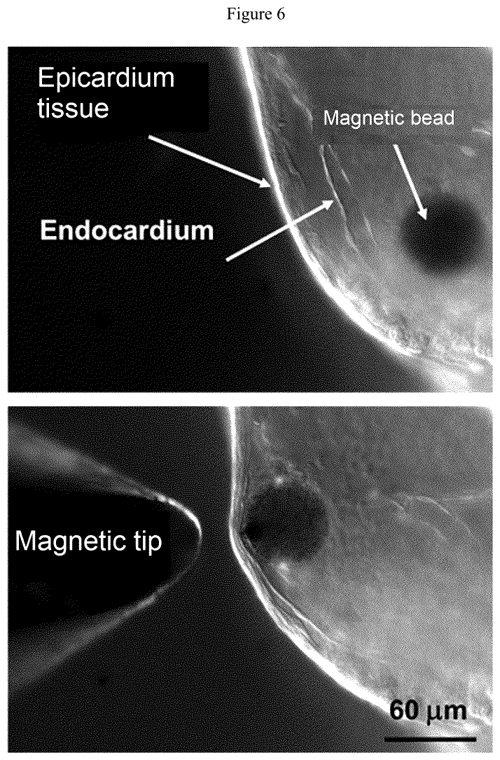

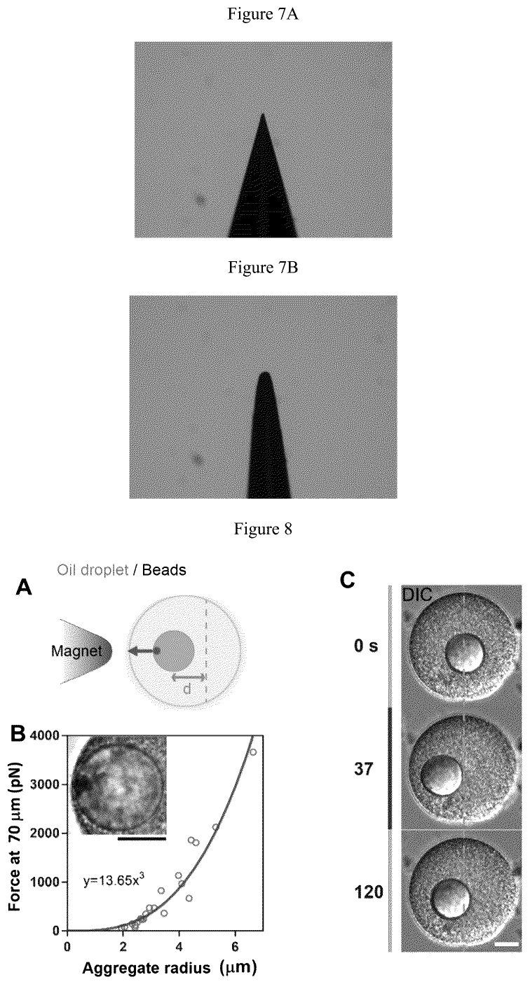

Tested and Validated Protocols
(i) Injection and displacement of streptavidin (Chemicell) coated 100-nm particles that do not exhibit specific interactions with the cellular structures used for calibration and control experiments (FIGS. 2 and 3).
(ii) Injection and displacement of magnetic particles coupled to quantum dots and 200 nm streptavidin (Nvigen) covered by purified molecular motors (dyneins) making it possible to target them to the microtubules and centrosome.
(iii) Injection and displacement of 1-μm diameter particles passivated with bovine serum albumin (BSA) (Dynabeads) allowing an inert displacement in the cytoplasm of the cells (FIG. 3).
(iv) Injection and displacement of 1-μm diameter beads (Dynabeads) associated with an anti-nuclear pore antibody (anti-NPC) making it possible to target them to the nuclear envelope and deform the nucleus. In particular, they were used to deform the nuclear membrane and measure its properties (FIG. 4).
(v) Injection and displacement of NanoLink (Solulink) brand 800-nm beads, which exhibit non-specific aggregation and adhesion properties to different biological structures. In particular, they were used to deform the cell membrane and measure its properties (FIG. 5).
(vi) Injection and displacement of very large 30-50 μm beads (Magne) to deform large portions of tissue within the heart of a zebrafish embryo (FIG. 6).

Imaging

The magnetic tip system is compatible with different types of microscopy. The images shown in FIGS. 2A, 3A-3C, 4 and 6 were taken on a wide-field fluorescent microscope (TI-Eclipse; Nikon) equipped with a CMOS camera (Orca-Flash4.0 LT; Hamamatsu). The samples were imaged with a 20×dry lens (NA, 0.75; Apo; Nikon) and a 1.5×magnifier. The microscope is controlled by Micro-Manager (Open Imaging). FIGS. 3B and 3C were taken on a confocal spinning head microscopy system, Yokogawa X1, mounted on a Nikon Ti stand, equipped with an EM-CCD camera. The cells are imaged with a Nikon 60X 1.4 NA Plan Apo water lens. The images are taken at room temperature (18-20° C.).

Results

A New Method for Applying Large Forces to Cellular or Nuclear Surfaces within Living Tissues.

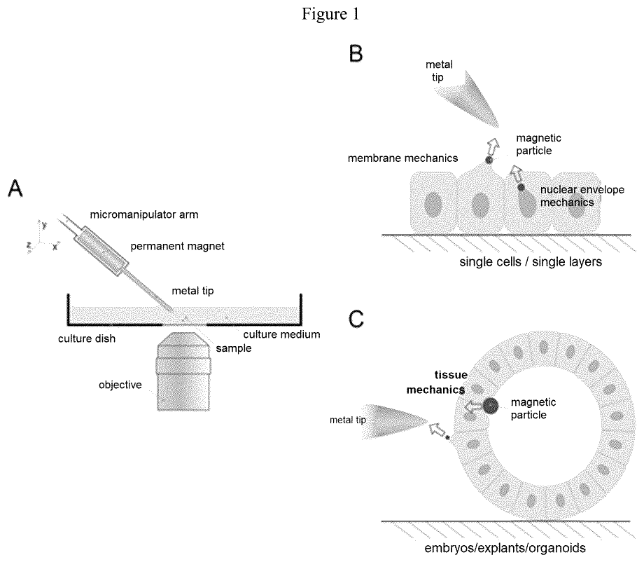

Currently, commercial methods of force applications on living cells, such as AFM (atomic force microscopy) or optical tips, remain unsuitable for deep samples, such as tissues or embryos, especially because of a limited force range (often below or around pN), and the lack of deep access to samples. Here, the inventors propose a method based on the use of magnetic tips, making it possible to apply calibrated forces to magnetic particles associated with the surface of a tissue, a single cell, or a nucleus within a cell (FIG. 1).

To establish the application possibilities of the magnetic tip system, the applicable forces were calibrated according to different magnetic beads, for distances ranging from ten to one hundred microns, corresponding to the scales associated with biological samples, from the single cell to the multicellular assemblies. For this purpose, a magnetic tip is coupled to a 3D micromanipulator and immersed in a viscous liquid of known viscosity. Fluorescent magnetic beads are placed in solution, and image sequences (films) are recorded to measure the velocity of the particles as a function of their distance from the magnetic tip (FIG. 2A). Different media with different viscosity values allow the calibration range to be extended over distances of up to several hundred microns (FIG. 2B). The velocity of the particle as a function of its distance to the tip is extracted through an ad hoc image analysis script, and makes it possible to calculate the magnetic force using Stokes' law: F=6πηRV, where η is the viscosity of the liquid, R is the radius of the bead and V is the velocity of the bead. By using different beads with sizes ranging from 1-50 μm, a force range from fraction of pN to a hundred nN, covering an amplitude range over 5-6 orders of magnitude, is demonstrated, thus establishing the versatility in terms of amplitude of forces applicable by this system (Table 1).

To validate the applicability of this method in deep samples, magnetic beads were then injected into single and multicellular samples with typical sizes of about 100 microns (FIG. 3A-3B). These experiments show that beads injected into these cells or into the lumen of large embryos can be displaced easily. Moreover, by videomicroscopy of cell divisions or embryo development over several hours (FIG. 3C-3D), the injected cells will be able to divide, and tissues will develop normally, suggesting that bead injection and manipulation do not affect the physiology of cells and tissues.

Application to Mechanical Measurements of the Surface of the Cell Nucleus.

One of the first applications of the method according to the invention is to probe the mechanical properties of the surface of the nucleus, within a living cell. This deep force application is totally incompatible with the available commercial methods mentioned above. This quantitative information is, however, very valuable because the properties of the nucleus have been suggested as markers of many processes such as cell differentiation, which are highly deregulated in cancer cells (Kirby and Lammerding, 2018). Nevertheless, measuring these properties within a living cell is a difficult task and has never been reported in the literature. To validate this approach, a protocol was established to attach, to the surface of magnetic beads, antibodies against nuclear pores, proteins highly conserved in eukaryotes. These beads injected into a cell can bind and accumulate specifically on the surface of the nucleus and allow the nucleus to be deformed by the approach of a magnetic tip. Measuring the deformation as a function of the applied force makes it possible to directly calculate an elasticity (stiffness) of the nuclear envelope. Thus, for example, in the experiment presented in FIG. 4, a force of 560.4 pN is applied and the resulting deformation is ˜8.7 m; this makes it possible to obtain an effective stiffness of the nuclear envelope 62.2 pN/m directly within a living cell.

Application to Mechanical Measurements on the Cell Surface on a Single Cell.

A second application of the method is in the calculation of the stiffness of the cell surface. The cell surface is limited by the plasma membrane that contains below it a cortex made of actin and myosin that is important for surface integrity. The properties of this cortex are also essential for processes such as cell migration or division that can be deregulated in tumour and metastatic cells, for example. To date, the mechanical properties of the cortex are thus a target for the study of fundamental mechanobiology and mechanobiology applied to medicine. These properties remain difficult to measure accurately, especially in deep tissues. By injecting magnetic beads into a cell and approaching them to the cell surface, forces can be directly applied to deform the cortex and measure its viscoelastic properties. Thus, from the experiment presented in FIG. 5, a stiffness of 16.7 pN/m and an effective viscosity of 1008.5 pN/m of the cell membrane are measured.

Application to Mechanical Measurements of Multicellular Tissues.

Another important application of the magnetic tip system is to be able to probe forces within tissues at depths of around a hundred micrometres. This aspect is essential, because modern disease study models (e.g., organoids, embryoids, cancerous spheroids) are 3D models with size scales of hundreds of microns, where the need to probe the properties of tissues in depth is substantial. For this purpose, the inventors demonstrate here the possibility of injecting large magnetic beads into the lumen of cardiac tissues of a vertebrate model embryo (zebrafish). These beads make it possible to apply tensile forces of around several tens of nN. When the beads are displaced by the magnetic tip, they compress the tissue and also deform it. These experiments thus make it possible to measure mechanical compression stiffness constants of 16.3 mN/m and a tissue surface elasticity of 12.1 mN/m, corresponding to an elasticity of ˜1.7 kPa, corresponding to an order of magnitude typical for cardiac tissues measured by other means (Kshitiz et al., 2012).

### Discussion

The inventors present here a simple, versatile and robust method of applying magnetic forces adapted to large samples such as tissues or embryos. The first advantage of this method compared to current commercial methods is that it is first and foremost compatible with all types of microscopes and micromanipulators available in standard research and development laboratories. The second advantage is that it covers a range of forces and distances much greater than other approaches, spread over 5-6 orders of magnitude, and with working distances of up to several hundred micrometres. This gives uniqueness to the approach presented here, as it opens up access to measurements in the volume of deep samples such as developing embryos, tissue explants and organoids that serve as new standards in biomedical research (Simian and Bissell, 2017).

The inventors demonstrate a series of potential applications to measure and study the surface mechanics of a cell nucleus, an individual cell and a tissue. This versatility is made possible by the use of different magnetic particles of different sizes and by modulations of their surface coverage. These methods provide quantifications within living cells or tissues, which cannot be obtained by commercially available methods. The simplicity and versatility of the system promises its application in therapeutic screening or routine diagnosis. For example, in the context of therapeutic screening, it is possible to directly measure the effect of a compound or drug on the mechanobiology of a living cell sample. To conclude, this measurement method, given its range of applicability, flexibility and simplicity, could be used routinely both to study the fundamentals of mechanobiology and to develop standard diagnostic methods for medicine.

### BIBLIOGRAPHY

Carey, S. P., T. M. D'Alfonso, S. J. Shin, and C. A. Reinhart-King. 2012. Mechanobiology of tumor invasion: engineering meets oncology. Crit Rev Oncol Hematol. 83:170-183.
Engler, A. J., S. Sen, H. L. Sweeney, and D. E. Discher. 2006. Matrix elasticity directs stem cell lineage specification. Cell. 126:677-689.
Jevtic, P., and D. L. Levy. 2014. Mechanisms of nuclear size regulation in model systems and cancer. Adv Exp Med Biol. 773:537-569.
Kirby, T. J., and J. Lammerding. 2018. Emerging views of the nucleus as a cellular mechanosensor. Nature Cell Biology. 20:373-381.
Kshitiz, M. E. Hubbi, E. H. Ahn, J. Downey, J. Afzal, D. H. Kim, S. Rey, C. Chang, A. Kundu, G. L. Semenza, R. M. Abraham, and A. Levchenko. 2012. Matrix rigidity controls endothelial differentiation and morphogenesis of cardiac precursors. Sci Signal. 5:ra41.
Ladoux, B., and R.-M. Mège. 2017. Mechanobiology of collective cell behaviours. Nature Reviews Molecular Cell Biology. 18:743-757.
Lim, C. T., A. Bershadsky, and M. P. Sheetz. 2010. Mechanobiology. J R Soc Interface. 7 Suppl 3:S291-293.
Makale, M. 2007. Cellular mechanobiology and cancer metastasis. Birth Defects Res C Embryo Today. 81:329-343.
Murthy, S. E., A. E. Dubin, and A. Patapoutian. 2017. Piezos thrive under pressure: mechanically activated ion channels in health and disease. Nat Rev Mol Cell Biol. 18:771-783.
Roca-Cusachs, P., V. Conte, and X. Trepat. 2017. Quantifying forces in cell biology. Nature Cell Biology. 19:742-751.
Simian, M., and M. J. Bissell. 2017. Organoids: A historical perspective of thinking in three dimensions. J Cell Biol. 216:31-40.

1-41. (canceled)

42. An in vitro or ex vivo method for characterizing at least one mechanobiological property of at least one element, at least one cell compartment, at least one cell or at least one group of cells in a biological sample, said method comprising:
a) introducing superparamagnetic beads into the biological sample, said beads comprising a coating capable of targeting the superparamagnetic beads to a cell element or compartment of interest or of penetrating a cell or group of cells,
b) applying a magnetic field to the superparamagnetic beads in order to apply a force to the biological sample,
c) measuring at least one mechanobiological property of the cell element or compartment in the biological sample,
said biological sample being a cell or a multicellular biological sample and
the magnetic field being applied by a magnetic tip, said magnetic tip comprising at least one millimetre or submillimetre sized magnet extended by a metal tip.

43. The method according to claim 42, wherein the coating is a hydrophobic coating.

44. The method according to claim 43, wherein the superparamagnetic beads comprising a hydrophobic coating are mixed with a biocompatible oil capable of causing the superparamagnetic beads to penetrate into a cell or a group of cells, before being introduced into the biological sample.

45. The method according to claim 42, wherein the coating is a biological coating capable of specifically targeting the superparamagnetic beads to a cell element or compartment of interest, a cell or a group of cells.

46. The method according to claim 45, wherein the superparamagnetic beads comprising a biological coating are mixed with a solution before being introduced into the biological sample.

47. The method according to claim 42, wherein said biological sample is a multicellular biological sample selected from a tissue, an organ or an embryo, said biological sample not being of human embryonic origin.

48. The method according to claim 42, wherein the force applied to the biological sample is between 0.001 pN and 5000 nN.

49. The method according to claim 42, wherein the distance between the magnetic tip and the biological sample is between 10 micrometres and 1 millimetre.

50. The method according to claim 42, wherein the method further comprises (i) a preliminary step in which the biological coating of the beads is selected according to the cell element or compartment of interest, (ii) a preliminary step of preparing the tip and/or superparamagnetic beads and/or (iii) a step of calibrating the tip and beads.

51. The method according to claim 42, wherein the mechanobiological properties are selected from rheology, kinematics, mechanical strength, elasticity, stiffness, plasticity and viscoelastic properties, and any combination thereof.

52. The method according to claim 42, wherein the coating of the superparamagnetic beads is a hydrophobic coating selected from butyl, methyl, ethyl, octyl, propyl or octadecyl.

53. The method according to claim 42, wherein the biocompatible oil is selected from a soya oil, a linseed oil, an oil of the (C15-C40) alkane type, and a fluorocarbon oil.

54. The method according to claim 42, wherein, the biological coating of the superparamagnetic beads is a protein coating, comprising at least one bait protein capable of recognizing a prey protein on the cell element or compartment of interest.

55. The method according to claim 54, wherein the biological coating of the superparamagnetic beads is an antibody or a lectin.

56. The method according to claim 54, wherein the biological coating of the superparamagnetic beads is an antibody selected from an anti-sodium potassium ATPase, anti-PMCA1, anti-Pan-cadherin, anti-E-cadherin, anti-N-cadherin, anti-ADAM22, anti-LRP4, anti-Fas (APO-1, CD95), anti-CD27, anti-EGFR, anti-NPC and anti-NUP98 antibody.

57. The method according to claim 42, wherein the solution is a buffer solution.

58. The method according to claim 42, wherein the cell element or compartment is the internal or external cell membrane of at least one cell of the biological sample.

59. The method according to claim 42, wherein the superparamagnetic beads have a size comprised between 100 nm and 100 μm.

60. The method according to claim 42, wherein, the superparamagnetic beads comprise a biological coating and wherein, in step a), the superparamagnetic beads are injected into a cellular compartment of the biological sample.

61. A kit for characterizing at least one mechanobiological property of at least one element, at least one cell compartment, at least one cell or at least one group of cells in a biological sample, said method comprising:
a) a magnetic tip comprising a magnetic core comprising a superposition of millimetre or submillimetre sized magnets extended by a steel tip,
b) superparamagnetic beads comprising a coating capable of targeting the superparamagnetic beads to a cell element or compartment of interest or penetrating a cell or group of cells,
c) optionally, a solution for suspending and injecting the superparamagnetic beads into a cell or cell tissue, and
d) optionally at least one solution for calibrating the superparamagnetic beads and the magnetic tip.
